(19) United States
(12) Patent Application Publication
(10) Pub. No.: US 2018/0005160 A1
(43) Pub. Date: Jan. 4, 2018

(54) DETERMINING AND ENHANCING PRODUCTIVITY

(71) Applicant: Microsoft Technology Licensing, LLC, Redmond, WA (US)

(72) Inventors: Warren D. Johnson, III, Sammamish, WA (US); Devon K. Baldwin, Federal Way, WA (US); Chantrelle Nielsen, Seattle, WA (US); Manjit S. Gill, Woodinville, WA (US)

(21) Appl. No.: 15/199,591

(22) Filed: Jun. 30, 2016

(51) Int. Cl. G06Q 10/06; A61B 5/01; A61B 5/00; A61B 5/0205; A61B 5/0476; A61B 5/053; A61B 5/021; A61B 5/024; A61B 5/08; A61B 5/091

(52) U.S. Cl. CPC G06Q 10/06398; A61B 5/02055; A61B 5/01; A61B 5/4266; A61B 5/0476; A61B 5/021; A61B 5/024; A61B 5/0816; A61B 5/091; A61B 2503/24; A61B 5/0531

(57) ABSTRACT

Techniques and technologies for determining and enhancing productivity are described. In at least some embodiments, a system for includes a processing component operatively coupled to a memory; a productivity analyzer at least partially disposed in the memory, the productivity analyzer including one or more instructions that when executed by the processing component perform operations including: receive productivity data associated with usage of one or more productivity tools by at least one user during a time period; receive biometric data associated with one or more biometric aspects of the at least one user during the time period; analyze one or more aspects of the productivity data and the biometric data; and determine based on the analysis at least one productivity-related operation intended to enhance at least one productivity metric of the at least one user.

### BACKGROUND

Modern enterprises of all sizes often employ tools that are intended to facilitate productivity. Common productivity tools include email applications, electronic calendaring applications, instant messaging applications, word-processing applications, and other suitable tools. Such tools may, for example, enable electronic messages (e.g. email, instant messages, etc.) to be exchanged, allow information to be shared and discussed, provide electronic calendaring capabilities for scheduling meetings, and enable other capabilities that improve a user's ability to perform productive activities. Through use of such productivity tools, communication and collaboration within modern enterprises may be significantly enhanced, thereby improving productivity. Although highly desirable results have been achieved using conventional productivity tools, there is room for further improvement.

### SUMMARY

In at least some embodiments, a system for determining and enhancing productivity includes a processing component operatively coupled to a memory; a productivity analyzer at least partially disposed in the memory, the productivity analyzer including one or more instructions that when executed by the processing component perform operations including: receive productivity data associated with usage of one or more productivity tools by at least one user during a time period; receive biometric data associated with one or more biometric aspects of the at least one user during the time period; analyze one or more aspects of the productivity data and the biometric data; and determine based on the analysis at least one productivity-related operation intended to enhance at least one productivity metric of the at least one user.

Similarly, in at least some implementations, a method for determining and enhancing productivity, comprises: receiving productivity data associated with usage of one or more productivity tools by at least one user during a time period; receiving biometric data associated with one or more biometric aspects of the at least one user during the time period; analyzing using one or more processing devices one or more aspects of the productivity data and the biometric data; and determining using one or more processing devices at least one productivity-related operation at least partially based on the analysis, the at least one productivity-related operation intended to enhance at least one productivity metric of the at least one user.

And in at least some implementations, a system for determining and enhancing productivity, comprises: circuitry configured for receiving productivity data associated with usage of one or more productivity tools by at least one user during a time period; circuitry configured for receiving biometric data associated with one or more biometric aspects of the at least one user during the time period; circuitry configured for analyzing one or more aspects of the productivity data and the biometric data; and circuitry configured for determining at least one productivity-related operation at least partially based on the analysis, the at least one productivity-related operation intended to enhance at least one productivity metric of the at least one user.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

The detailed description is described with reference to the accompanying figures. In the figures, the use of the same reference numbers in different figures indicates similar or identical components.

FIG. 1 shows an embodiment of an environment for determining and enhancing productivity.

FIG. 2 shows an embodiment of a process for determining and enhancing productivity.

FIG. 3 shows an embodiment of a set of productivity metrics.

FIG. 4 shows another embodiment of an environment for determining and enhancing productivity.

FIG. 5 shows an embodiment of a system for determining and enhancing productivity.

FIG. 6 shows another embodiment of a process for determining and enhancing productivity.

FIG. 7 shows an embodiment of a computer system environment for gesture-controlled piling and un-piling of displayed data.

### DETAILED DESCRIPTION

The present disclosure describes techniques and technologies for determining and enhancing productivity. As described more fully below, techniques and technologies for determining and enhancing productivity in accordance with the present disclosure may advantageously provide substantial operational improvements in the operations of one or more computers operated by one or more users of an environment in comparison with conventional technologies. For example, techniques and technologies for determining and enhancing productivity in accordance with the present disclosure may advantageously enable users to at least partially mitigate distractions that may otherwise cause them to use their computers and other devices (or the one or more productivity tools operating on their computers and other devices) inefficiently. The resulting improvements in productivity may advantageously result in one or more tasks being performed on a device by the user to be performed more efficiently, using fewer computational operations, fewer computational processing cycles, and less energy consumption (e.g. less battery power) in comparison with conventional techniques.

As noted above, usage of modern productivity tools significantly enhances an organization's productivity. The ability to easily and quickly prepare communications to other workers, to share and discuss information, to organize and conduct meetings, and to perform various other tasks using modern productivity tools enables people of modern enterprises to communicate and collaborate with unprecedented ease and efficiency. It will be appreciated, however, that although such productivity tools provide substantial benefits, the benefits realized through usage of modern productivity tools may be further enhanced by techniques and technologies that appropriately balance the use of such productivity tools based on various factors, such as an individual user's goals, health, responsibilities, personal characteristics or other suitable factors.

For example, in at least some implementations, techniques and technologies for determining and enhancing productivity as disclosed herein may analyzing one or more of productivity data, biometric data, line of business data, or individual goals data, and from these analyses, determine one or more productivity-related operations intended to promote or enhance productivity. As used herein, the term “biometric data” refers to digital data resulting from the capture or sensing of one or more characteristics of a living entity. For example, based on analysis a computer user's biometric data and productivity data regarding the user's usage of the computer, and a correlation may be determined, and based on the correlation, a productivity-related operation that enhances the user's usage of the computer may be determined and performed. Such productivity-related operations may include, for example, adjusting one or more aspects of a user's productivity tool(s), adjusting one or more aspects of displayed items, providing one or more notifications intended to improve productivity, providing one or more haptic prompts intended to improve productivity, or other suitable productivity-related operations. In this way, techniques and technologies in accordance with the present disclosure may provide substantial operational improvements in the operations of one or more computers operated by one or more users of an environment in comparison with conventional technologies, as described more fully below.

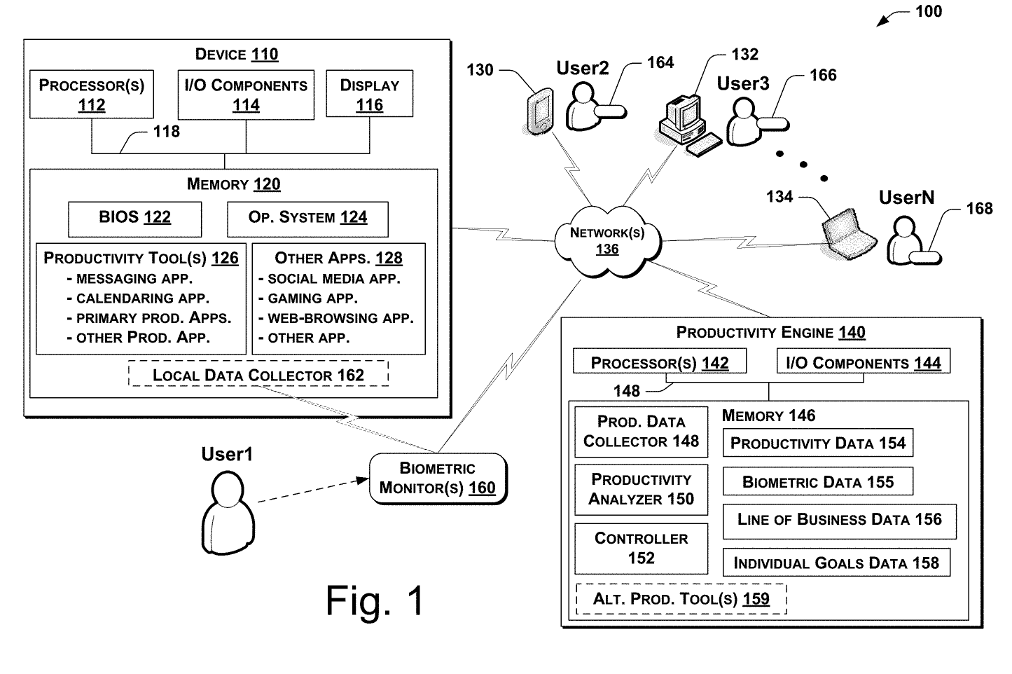

FIG. 1 shows an embodiment of an environment 100 for determining and enhancing productivity in accordance with the present disclosure. In this embodiment, the environment 100 includes a device 110 associated with a first user (User1), the device 110 having one or more processing components 112, one or more input/output (I/O) components 114, and a display 116 operatively coupled to a memory 120 by a bus 118. The memory 120 of this embodiment includes a basic input/output system (BIOS) 122, which provides basic routines that help to transfer information between elements within the device 110, and an operating system 124 that manages and provides common services to the various elements of the device 110. In the embodiment shown in FIG. 1, the device 110 further includes one or more productivity tools 126, and one or more other (or non-productivity) applications 128 loaded on the memory 120. In some implementations, a local data collector 162 may also be stored on the memory 162.

In at least some implementations, the one or more productivity tools 126 may include one or more of an electronic messaging application (e.g. email, instant messages, etc.), an electronic calendaring application, one or more primary productivity applications, or any other productivity application. In at least some implementations, the one or more primary productivity application may be an application(s) that enables a user to accomplish their primary work-place responsibilities, such as a word-processing application (e.g. Microsoft Word®), an application for creating drawings (e.g. Microsoft Visio®), a spreadsheet application (Microsoft Excel®), a presentation application (e.g. Microsoft PowerPoint®), a computer-aided design (CAD) application, or any other suitable productivity tools. In addition, in at least some implementations, the one or more productivity tools 126 may be packaged or combined into a single application or suite of applications. For example, in at least some implementations, the messaging and calendaring capabilities may be combined in to a single application suite, such as the Microsoft Outlook® product.

The one or more other applications 128 may generally include any applications that are not categorized as one of the productivity tools 126. For example, in at least some implementations, the one or more other applications 128 may include a social media application (e.g. Facebook, Twitter, Snapchat, etc.), a gaming application, a web-browsing application (e.g. Internet Explorer®, Chrome®, Firefox®, etc.), or any other type of non-productivity application. It will be appreciated that the one or more productivity tools 126 and the other applications 128 are rigidly defined and are not mutually exclusive, and that for some users on some devices, an application may be a productivity tool 126 (e.g. web-browsing application, social media application, etc.), while for other users, the same application may be considered a non-productivity application 128.

In the representative environment 100 shown in FIG. 1, the device 110 is operable to communicate with other user devices (e.g. devices 130, 132, 134) associated with other users (e.g. User2, User3, UserN) via one or more networks 136. In addition, a productivity engine 140 is operatively coupled to the one or more networks 136 to perform one or more aspects of the techniques for determining and enhancing productivity in accordance with the present disclosure, as described more fully below. In the environment 100 depicted in FIG. 1, there is a single device associated with each user (e.g. device 110 associated with User1, device 130 associated with User2, etc.) for the sake of clarity, however, it will be appreciated that in alternate implementations, a suitable environment may have multiple devices associated with one or more of the users.

It will be appreciated that the device 110 (and devices 130, 132, 134) shown in FIG. 1 may represent a variety of possible device types, including but not limited to a personal computer, a laptop computer, a handheld device, such as a cellular telephone, a Personal Data Assistant (PDA), a notebook computer, a tablet computer, a slate computer, a smart watch, or any other handheld device. It should be understood, however, that the device 110 (or devices 130, 132, 134) is not limited to these particular example devices, and may represent a server, a mainframe, a workstation, a distributed computing device, a portion of a larger device or system (e.g. a control component of a distributed computing device), or any other suitable type of device. In still other embodiments, the device 110 (or devices 130, 132, 134) may be a television, a wearable device, a vehicle (or portion of a vehicle), an appliance (or portion of an appliance), a consumer product, a component of the Internet of Things, or virtually any other suitable device.

One or more biometric monitors 160 are operatively associated with the first user (User1) to record biometric data regarding one or more biometric aspects of the first user (User1). For example, in at least some implementations, the one or more biometric monitors 160 may collect data regarding one or more of respiration (e.g. rate, volume, duration, pattern, etc.), heart rate, blood pressure, temperature, perspiration, skin conductivity, brain activity data (e.g. brain waves, brain temperature data, electroencephalogram (EEG) etc.), or any other suitable biometric aspects of the first user (User1). At least some of the one or more biometric monitors 160 may be worn by (or in contact with) the first user (User1), or alternately, may be operatively positioned in the vicinity of the first user (User1) to sense biometric data in a non-contacting manner. The one or more biometric monitors 160 may be any of a variety of generally-known devices for sensing one or more characteristics of the first user (User1). For example, the one or more biometric monitors 160 may include one or more of the devices commercially-available from Spire, Inc., Fitbit, Inc., Jawbone, Inc., Garmin, Inc., Apple, Inc., Adidas, Inc. and a variety of other suitable devices.

In some implementations, the one or more biometric monitors 160 may transmit at least some of the collected biometric data via the one or more networks 136 to the productivity engine 140. In some other implementations, the one or more biometric monitors 160 may transmit at least some of the collected biometric data to a local data collector 162 of the device 110, whereupon the device 110 may transmit the collected biometric data from the local data collector 162 to the productivity engine 140 via the one or more networks 136 at a suitable time (e.g. periodically, non-periodically, upon satisfaction of a condition, upon the device 110 reconnecting to the one or more networks 136, etc.). Similarly, the environment 100 further includes one or more biometric monitors 164 operatively associated with a second user (User2), one or more biometric monitors 166 operatively associated with a third user (User3), and one or more biometric monitors 168 operatively associated with an n^{th} user (UserN).

With continued reference to FIG. 1, in at least some implementations, the productivity engine 140 includes one or more processors 142 and one or more I/O components 144 operatively coupled to a memory 146 by a bus 148. In at least some implementations, the memory stores 146 stores a productivity data collector 148, a productivity analyzer 150, and a controller 152. Similarly, in at least some implementations, the memory 146 may also host one or more of productivity data 154, biometric data 155, line of business data 156, or individual goals data 158. In at least some implementations, one or more alternate productivity tools 159 may be installed on the productivity engine 140 for access and usage by one or more of the users (User1, User2, User3, User4) of the environment 100 via the one or more networks 136 (e.g. as in a cloud-computing environment, a centralized computing environment, etc.). In at least some implementations, the one or more alternate productivity tools 159 may be substantially similar to, or substantially the same as, the one or more productivity tools 126 described above, allowing one or more of the users of the environment 100 to access and use the alternate productivity tools 159 via one or more alternate devices that may not have the one or more productivity tools 126 installed thereon.

The productivity data collector 148 is operable to collect and store the productivity data 154, and the productivity analyzer 150 is operable to access and analyze one or more of the productivity data 154, the biometric data 155, the line of business data 156, or the individual goals data 158. Based on the analysis of the productivity analyzer 150, the controller 152 may perform one or more control operations in accordance with one or more aspects of techniques for determining and enhancing productivity in accordance with the present disclosure, as described more fully below.

More specifically, in at least some implementations, the productivity data collector 148 may obtain data regarding the usage of the productivity tools 126 on the device 110 by the first user (User1), and may also obtain data regarding the usage of productivity tools on the other devices (e.g. 130, 132, 134) by the other users (User, User3, UserN). In at least some implementations, the productivity data collector 148 may monitor or query the usage of the one or more productivity tools 126 to obtain at least some of the productivity data 154, or may receive at least some of the productivity data 154 from the local data collector 162, or any suitable combinations thereof. The productivity data collector 148 stores the collected productivity data 154 on the memory 146 of the productivity engine 140. In at least some implementations, the productivity data collector 148 (and/or the local data collector 162) may also collect and store data regarding the usage of the other (or non-productivity) applications 128 (e.g. social media application, gaming application, web-browsing application, etc.).

In at least some implementations, the line of business data 156 represents an organizational or managerial hierarchy of the users (e.g. User1, User2, User3, UserN) within the environment 100. For example, in at least some implementations, the line of business data 156 may indicate that some of the users are on an equal (or substantially equal) level of responsibility within an organization, while other users may have managerial responsibility over some other the other users. In further implementations, the line of business 156 data may establish a hierarchy of the users of the environment in other ways, such as age, seniority, occupation, subscription level, volume or rate of messaging or other suitable metric, or any other suitable way. The line of business data 156 may be established in a variety of ways, such as by being input or updated by an administrator of the productivity engine 140, or by one or more users within the environment 100 having authority to input or update the line of business data 156 (e.g. a manager, system administrator, executive, etc.), such as to reflect employee positions, promotions or changes of responsibility, etc.

For example, in one representative embodiment, the line of business data 156 may indicate that the first user (User1) and the second user (User2) are on a substantially equal level of the managerial hierarchy of an organization (e.g. equal pay grade, equal job title, equal seniority level, etc.), while the third user (User3) may have managerial responsibility over the first and second users (User1, User2), and the n^{th} user (UserN) may be the top executive (or highest level manager) within the organization, with managerial authority over all other users (User1, User2, User3). In such a representative embodiment, the productivity analyzer 150 may take into consideration the relative equality of the first and second users (User1, User2), the relative authority of the third user (User3) over the first and second users (User1, User2), and the relative authority of the n^{th} user (UserN) over all other users (User1, User2, User3) while analyzing the various data (154, 155, 156, 158), such as in the prioritization of calendared meetings, the anticipated impact of electronic messages, or during analysis of aspects in the biometric data 155 from the biometric monitors (160, 162, 164, 166), or in other possible ways, as described more fully below.

Referring again to FIG. 1, in at least some implementations, the individual goals data 158 may be input by the users (User1, User2, User3, UserN) of the environment 100, and may represent each user's individual goals for achieving an appropriate balance of one or more specified work and personal health characteristics. Alternately, in at least some implementations, at least some of the individual goals data 158 may be established in other ways, such as by default settings, or prescribed by an appropriate authority (e.g. a manager of the user, by a policy of the user's organization, etc.). For example, in at least some implementations, the individual goals data 158 may include a user's individualized goals for achieving an appropriate balance of work-related activities and health-related activities. More specifically, in one representative embodiment, the individual goals data 158 may specify a user's individual goals for a weekly cycle in terms of work-related activities, such as time spent in meetings, and time spent using one or more productivity tools 126 (e.g. time spent emailing or number of emails, time spent word or volume of word processing performed, time spent writing code or volume of code written, etc.). The individual goals data 158 may also specified the user's weekly goals for personal health-related activities, such as time spent or volume of exercising (e.g. number of steps taken, number of reps performed, number of minutes of elevated heart or respiratory activity), recreational activities (e.g. time spent or volume of gaming, time spent away from screens, etc.) or one or more other individual goals or metrics.

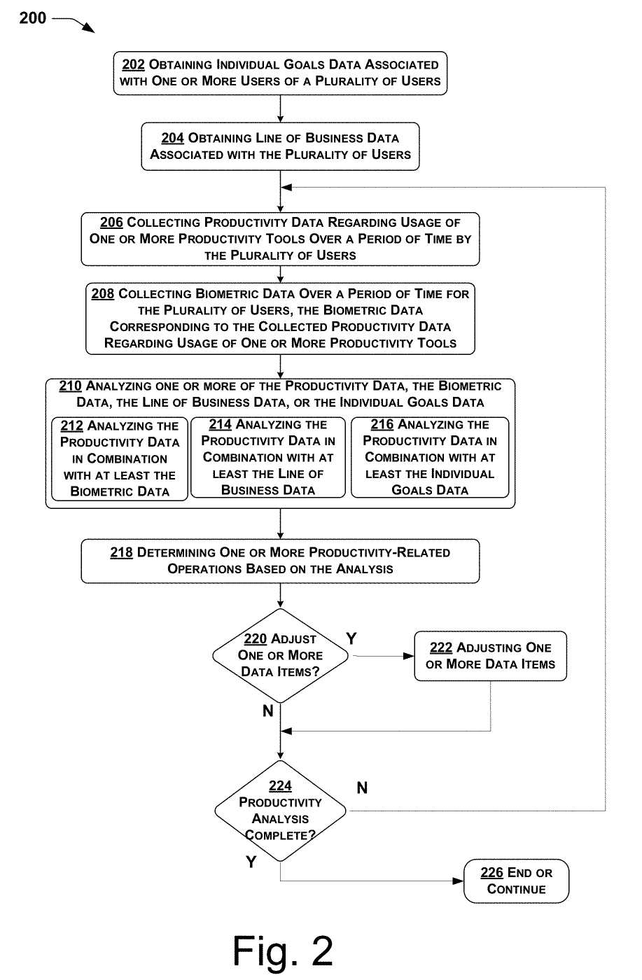

FIG. 2 shows an embodiment of a process 200 for determining and enhancing productivity in accordance with the present disclosure. In general, the process 200 may be performed by a device or system (e.g. productivity engine 140) appropriately configured to perform the described operations. In the embodiment shown in FIG. 2, the process 200 includes obtaining individual goals data associated with one or more users of a plurality of users at 202. For example, in at least some implementations, one or more of the users (User1, User2, User3, UserN) of the environment 100 (FIG. 1) may input their own individual goals data via their respective devices (110, 130, 132, 134), which in turn may be collected by the productivity engine 140 (e.g. by the productivity data collector 148) and stored within the individual goals data 158. Alternately, at least a portion of the individual goals data 158 may be input by an administrator, or established by defaults or policies associated with the users of the environment 100.

In the embodiment shown in FIG. 2, the process 200 further includes obtaining line of business data associated with the plurality of users at 204. For example, in at least some implementations, the productivity engine 140 may receive inputs from a system administrator or from one or more of the users (User1, User2, User3, UserN) of the environment 100 (e.g. via the productivity data collector 148) for storage as the line of business data 156. As noted above, in at least some implementations, the line of business data 156 may indicate a structural or hierarchical relationship (e.g. managerial, seniority based, etc.) of the users within an organization represented by the environment 100.

As further shown in FIG. 2, the process 200 for determining and enhancing productivity further includes collecting productivity data regarding usage of one or more productivity tools over a period of time by the plurality of users at 206. For example, in at least some implementations, as the first user (User1) uses one or more of the productivity tools 126 installed on the device 110, or one or more of the alternate productivity tools 159 installed on the productivity engine 140, data regarding such usage by the first user (User1) may be collected (at 206) by the productivity data collector 148 and stored within the productivity data 154 of the productivity engine 140 (e.g. time spent in meetings as indicated by electronic calendar data, time spent preparing messages or reviewing messages as indicated by electronic messaging data, time or volume of word processing, time or volume of coding performed, etc.). The productivity data 154 may therefore include data for the first user (User1) which may be processed and analyzed to determine various metrics related to productivity of the first user (User1), as described more fully below.

In at least some implementations, the collecting of productivity data (at 206) may be performed during specified periods of a day or during specified periods that are typically considered as work time, while in at least some other implementations, the collecting (at 206) may be performed round-the-clock or continuously. In addition, in at least some implementations, the collecting of productivity data (at 206) may be performed for all of the users of the environment 100 (e.g. User1, User2, User3, UserN), while in alternate implementations, the collecting of the productivity data (at 206) may be performed for only a subset or portion of the users of the environment 100 (e.g. only the first, second, and third users (User1, User2, User3) but not for a top executive user (UserN)).

Additionally, in at least some implementations, the collecting of productivity data (at 206) may include the collection of data regarding the usage of the one or more other (or non-productivity) applications 128 by the first user (User1). For example, in at least some implementations, as the first user (User1) uses one or more of the other applications 128 installed on the device 110, data regarding such usage by the first user (User1) may be collected by the productivity data collector 148 and stored within the productivity data 154. Again, in at least some implementations, the collecting of productivity data (at 206) that includes usage data for one or more other applications 128 may be performed for all of the users of the environment 100 (e.g. User1, User2, User3, UserN), or alternately, may be performed for only a subset of the users of the environment 100.

Referring again to FIG. 2, in the depicted embodiment, the process 200 further includes collecting biometric data over a period of time for the plurality of users at 208. More specifically, in at least some implementations, the collecting of biometric data (at 208) may include collecting biometric data over a period of time that at least partially corresponds (or overlaps) with the collection of productivity data over a period of time (at 206). For example, in at least some implementations, the collecting of productivity data (at 206) may be performed during a specified period of a day that a user specifies as a work-related period (e.g. for 10 hours beginning at 8:00 am), while the collecting of biometric data (at 208) may be performed during another specified period of the day that overlaps with the period of productivity data collection (e.g. for 16 hours beginning at 7:00 am). Thus, for at least some implementations, the productivity data 154 (collected at 206) and the biometric data 155 (collected at 208) may be processed and analyzed to determine whether any correlations exist between these data, as described more fully below. In at least some implementations, the collecting of biometric data (at 208) may be performed during times or periods that may typically be considered non-working times (e.g. during evenings and weekends), as well as during times or periods normally considered as working times, so that the biometric data may be processed and analyzed to determine various individual goals established by the users (e.g. weekly time spent or volume of exercising, daily number of steps taken, weekly number of reps performed, number of minutes of elevated heart or respiratory activity per month, etc.). Of course, in at least some implementations, the collecting of biometric data (at 208) may be performed round-the-clock or continuously. In addition, in at least some implementations, the collecting of biometric data (at 208) may be performed for all of the users of the environment 100, while in alternate implementations, the collecting of the biometric data (at 208) may be performed for only a subset of the users of the environment 100.

The process 200 further includes analyzing one or more of the productivity data, the biometric data, the line of business data, or the individual goals data at 210. More specifically, in at least some implementations, the analysis (at 210) may include the productivity analyzer 150 of the productivity engine 140 analyzing the productivity data 154 to determine one or more productivity metrics. For example, the productivity data 154 may be processed and analyzed to determine aggregate amounts of time spent by one or more of the users (e.g. the first user User1) in meetings, sending and receiving messages, or operating one or more primary productivity tools (126, 159) during a given period (e.g. daily, weekly, bi-weekly, etc.). Similarly, the productivity data 154 may be processed and analyzed to determine other aggregated metrics, such as a volume of messages sent, a volume of word-processing performed, a volume of other productivity indicia performed (e.g. hours of coding, lines of coding, number of messages drafted, pages of documentation reviewed, etc.) during a given period of time.

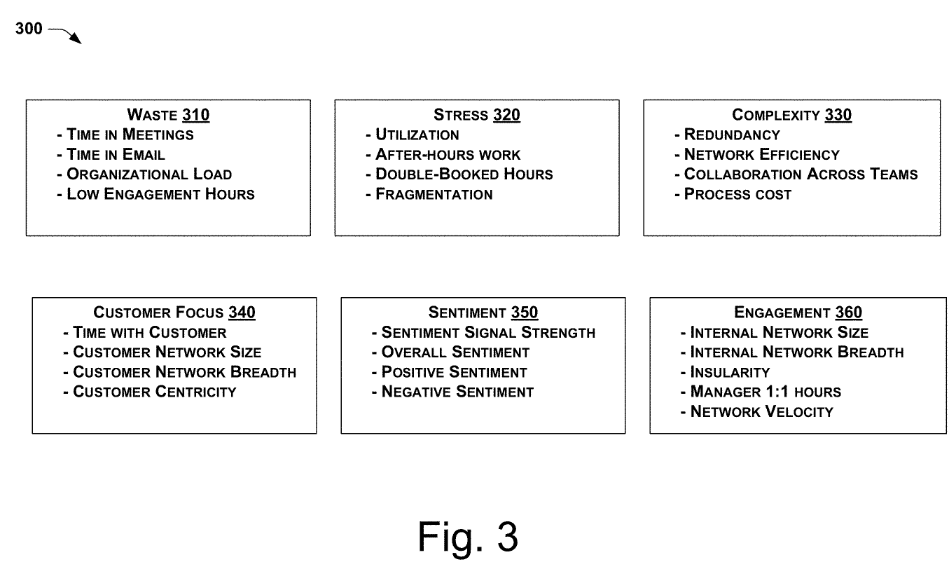

FIG. 3 shows an embodiment of a set of productivity metrics 300. In this embodiment, the productivity metrics 300 include six categories of productivity metrics 300 that may be determined during the analysis (at 210). In the embodiment shown in FIG. 3, the productivity metrics 300 include waste 310, stress 320, complexity 330, customer focus 340, sentiment 350, and engagement 360. More specifically, in at least some implementations, the waste 310 productivity metric may include one or more of time spent in meetings (e.g. time spent in calendared business meetings, excludes personal and social appointments, double-booked time, and time blocked on the calendar for independent work), time spent in email (e.g. time spent sending and receiving email, estimated as 5 minutes per sent email and 2.5 minutes per received email, adjustable as desired), organizational load (e.g. amount of time that an individual took from the rest of the organization based on emails they sent and meetings they scheduled), or low engagement hours (e.g. time spent in meetings but “disengaged,” defined as the average of (a) redundant time, (b) double-booked time, and (c) time in a meeting spent sending emails, such as at least two emails per hour).

Similarly, in at least some implementations, the stress 320 productivity metric may include one or more of utilization (e.g. the effective length of the work week, measured by the duration between the first and last email or meeting of the day, may be capped at 80 hours M-F), after-hours work (e.g. time spent on email and meetings outside normal business days and hours, M-F 8 am-5 pm), double-booked hours (hours per week where the individual had two meetings scheduled at the same time, only “business-relevant” meetings are counted, not personal time blocked on the calendar), or fragmentation (e.g. counts the “flow time” available to a person to get work done, defined as two-hour blocks of time that are uninterrupted by meetings). In at least some implementations, the complexity 330 productivity metric may include one or more of redundancy (e.g. meeting time in which there were at least three layers of management present from within a single function), network efficiency (e.g. average amount of time spent with each “strong ties” connection inside the organization, less time per connection indicates a network that is efficient for finding information and getting things done), collaboration across teams (e.g. the percentage of any team's total time that is spent with other specified teams), or process cost (e.g. the cost of time spent in meetings and email corresponding to a set of keywords and/or group participation rules).

In at least some implementations, the customer focus 340 productivity metric may include one or more of time with customer (or external collaboration time) (e.g. percentage of total meeting and email time spent with external people, possible to tag and target any sub-group of external people), customer network size (e.g. number of distinct external people with with each person maintained ties per month), customer network breadth (e.g. number of connections there have been with domains outside of your company over a selected time period, determined by the domain of the email address “@companyX.com” of the person contacted), or customer centricity (e.g. how central a person is to the flow of information within a company, a high centrality means that a person has more connections, and the people that they are connected to also tend to have many connections).

In at least some implementations, the sentiment 350 productivity metric may include one or more of sentiment signal strength (e.g. the signal strength of all words with any emotional content that are present in email and meeting subject lines sent by a user), overall sentiment (e.g. the weighted average sentiment score of the words present in email and meeting subject lines sent by a user, not on a percent scale), positive sentiment (e.g. the proportion of positive words present in email and meeting subject lines sent by a user), or negative sentiment (e.g. the proportion of negative words present in email and meeting subject lines sent by a user). And in at least some implementations, the engagement 360 productivity metric may include one or more of internal network size (e.g. number of “strong ties” connections a person maintains in a month, connections of at least two emails or meetings with fewer than five people), internal network breadth (e.g. number of departments per month in which a person maintains “strong ties” connections), insularity (e.g. the percentage of activity for the group that involved only members of the same group, a “group” can be department, function, location, etc.), manager 1:1 hours (e.g. the average amount of time per week a person spends in 1:1 meetings with his or her supervisor), or network velocity (e.g. the pace at which new strong-ties connections are added every month within the organization).

As further shown in FIG. 2, in at least some implementations, the analyzing one or more of the productivity data, the biometric data, the line of business data, or the individual goals data (at 210) may include analyzing the productivity data in combination with at least the biometric data at 212. For example, in at least some implementations, the productivity analyzer 150 may determine one or more correlations between one or more aspects of the productivity data 154 and one or more aspects of the biometric data 155. Similarly, in at least some implementations, the analyzing one or more of the productivity data, the biometric data, the line of business data, or the individual goals data (at 210) may include analyzing the productivity data in combination with at least the line of business data at 214. For example, in at least some implementations, the productivity analyzer 150 may determine one or more correlations between one or more aspects of the productivity data 154 and one or more aspects of the line of business data 156. Alternately, in at least some implementations, the productivity analyzer 150 may determine one or more correlations between one or more aspects of the productivity data 154, one or more aspects of the biometric data 155, and one or more aspects of the line of business data 156.

With continued reference to FIG. 2, in at least some implementations, the analyzing one or more of the productivity data, the biometric data, the line of business data, or the individual goals data (at 210) may include analyzing the productivity data in combination with at least the individual goals data at 216. For example, in at least some implementations, the productivity analyzer 150 may determine one or more correlations between one or more aspects of the productivity data 154 and one or more aspects of the individual goals data 158. Alternately, in at least some implementations, the productivity analyzer 150 may determine one or more correlations between one or more aspects of the productivity data 154, one or more aspects of the biometric data 155, and one or more aspects of the individual goals data 158. And in at least some further implementations, the productivity analyzer 150 may determine one or more correlations between one or more aspects of the productivity data 154, one or more aspects of the biometric data 155, one or more aspects of the line of business data 156, and one or more aspects of the individual goals data 158.

In the embodiment shown in FIG. 2, the process 200 for determining and enhancing productivity includes determining at 218 one or more productivity-related operations based on the analysis (at 210). In general, the determining one or more productivity-related operations (at 218) may include adjusting one or more aspects of a user's usage of the one or more productivity tools (126, 159), adjusting one or more aspects of displayed items to improve productivity, providing one or more notifications to the user containing suggestions or recommendations intended to improve productivity, providing one or more haptic prompts to intended to improve productivity, or other suitable productivity-related operations.

For example, in at least some implementations, the determining one or more productivity-related operations (at 218) may include adjusting one or more aspects of a display of an upcoming meeting on an electronic calendar application to indicate whether or not the user's attendance at the meeting would be consistent with the one or more aspects of a user's productivity, such as the user's individual goals data 156. More specifically, if the analysis (at 216) indicates that an upcoming meeting appearing on the user's electronic calendar appears to be consistent with the user's productivity (e.g. individual goals data 156), the appearance of the meeting in the user's electronic calendar may be displayed in a first manner (e.g. with a white background, with a green indicator, etc.) indicating that the user is encouraged or recommended to attend the meeting. On the other hand, if the analysis (at 216) indicates that the upcoming meeting will be inconsistent with the user's productivity (e.g. based on past event history, the meeting value is low and the time to return to productivity is long), the appearance of the meeting in the user's electronic calendar may be displayed in a second manner (e.g. with a dark background, with a red indicator, etc.) indicating the user is discouraged or not recommended to attend the meeting.

Alternately, in at least some implementations, the determining one or more productivity-related operations (at 218) may include delaying delivery of one or more electronic messages (e.g. email messages, instant messages, etc.) if such delaying of electronic messages would be consistent with the one or more aspects of a user's productivity, such as the user's individual goals data 156. More specifically, if the analysis (at 216) indicates that the user has already spent considerable time reviewing electronic messages and that receiving additional messages would be inconsistent with the user's productivity (e.g. individual goals data 156), one or more new messages may be delayed from being delivered so that the user can perform other tasks (e.g. spend time with the primary productivity tool, time coding, time exercising, time away from screens, etc.) that are consistent with the user's productivity. More specifically, the possible delaying of electronic messages may be dependent upon various factors, such as whether the electronic messages are from persons of higher authority (e.g. based on the line of business data 156), or whether the messages have been indicated as being high importance (e.g. marked with red flag, or indicated as high priority in a subject line or header of the message, etc.), or based on whether the message is personal or business related (e.g. based on an identity of the sender, based on content in a subject or header of the message, etc.), or based on any other suitable factor.

Furthermore, in at least some implementations, the determining one or more productivity-related operations (at 218) may include providing an output that results in a notification to a user of a productivity-related event. For example, in at least some implementations, the notification to a user of a productivity-related event may include providing a notification (e.g. a pop up window, an electronic message, a text, an audible message, an automated call, etc.) to the user indicating that a certain threshold (e.g. a goal, a pre-established limit, target, etc.) has been reached regarding an aspect of the user's individual goals data 158 (e.g. time spent reviewing electronic messages per day, time spent using web-browsing application per week, time spent gaming, goal reached regarding exercise or movement, etc.). In at least some implementations, the notification may be a written message, or alternately, may include a non-visually based notification (e.g. audible notification, haptic notification, etc.).

Referring again to FIG. 2, in this embodiment, the process 200 further includes determining whether to adjust one or more data items at 218. For example, in at least some implementations, the determination (at 218) may include determining whether to adjust one or more of a user's individual goals data 158. Alternately, the determination (at 218) may include determining whether to adjust one or more items of the line of business data 156. Similarly, the determination (at 218) may include determining whether to change one or more aspects of the productivity data being collected (at 206), or whether to change one or more aspects of the biometric data being collected (at 208).

If it is determined that it is desirable to adjust one or more data items (at 218), then the process 200 proceeds to adjusting the one or more data items at 222. As noted above, the adjusting (at 222) may include, for example, one or more of adjusting one or more of a user's individual goals data 158, adjusting one or more items of the line of business data 156, changing one or more aspects of the productivity data being collected, changing one or more aspects of the biometric data being collected, or performing any other suitable adjustments.

If it is determined (at 218) that it is not desirable to adjust one or more data items, or after the adjusting of the one or more data items (at 222), the process 200 includes determining whether the productivity analysis is complete at 224. If the process 200 is not complete (at 224), then in the embodiment shown in FIG. 2, the process 200 returns to collecting productivity data (at 206), and the above-described operations 206 through 224 may be repeated one or more additional cycles, thereby continuing to determine and enhance productivity in accordance with the present disclosure. If the process 200 is determined to be complete (at 224), then the process 200 may end or continue to other operations at 226.

It will be appreciated that techniques and technologies for determining and enhancing productivity as disclosed herein may provide substantial operational improvements in the operations of one or more computers operated by one or more users of an environment in comparison with conventional technologies. For example, techniques and technologies for determining and enhancing productivity in accordance with the present disclosure may advantageously enable users to at least partially mitigate distractions that may otherwise cause them to use their computers and other devices (or the one or more productivity tools operating on their computers and other devices) inefficiently. For example, embodiments of systems and methods that reduce the number of interruptions that a user experiences while the user is operating one or more productivity tools (126, 159), may advantageously result in one or more tasks being performed on a device by the user to be performed more efficiently, using fewer computational operations, fewer computational processing cycles, and less energy consumption (e.g. less battery power) in comparison with conventional techniques wherein the user is less efficient due to increased interruptions or distractions from their productivity objectives. These improvements in efficiency may further translate into less wear and tear on processors, display components, circuitry, battery, and other components of devices and systems, thereby prolonging useful life and operability of such systems.

Techniques and technologies for determining and enhancing productivity in accordance with the present disclosure are not necessarily limited to the particular embodiments described above with reference to FIGS. 1-3. In the following description, additional embodiments of techniques and technologies for determining and enhancing productivity will be described. It should be appreciated that the embodiments described herein are not intended to be exhaustive of all possible embodiments in accordance with the present disclosure, and that additional embodiments may be conceived based on the subject matter disclosed herein. For example, it should be appreciated that at least some of the various components and aspects of the described embodiments may be eliminated to create additional embodiments, or may be variously combined or re-ordered to create still further embodiments. In the following discussion of additional embodiments, common reference numerals may be used to refer to elements introduced above, and for the sake of brevity, descriptions of previously-introduced elements may be omitted so that emphasis can be properly placed on new or varying aspects of such additional embodiments.

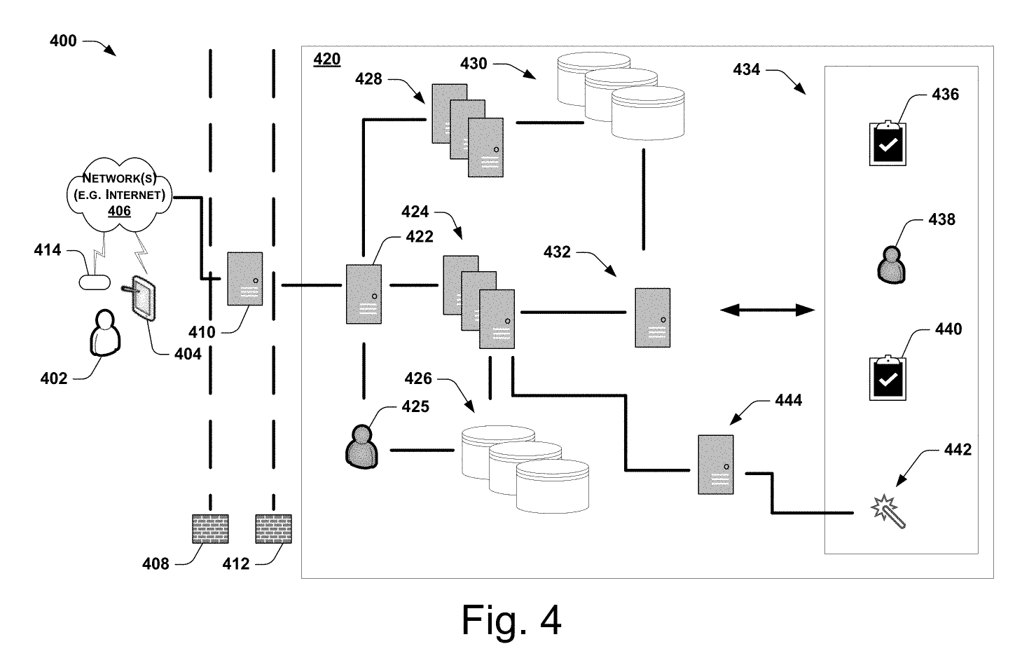

For example, FIG. 4 shows another embodiment of an environment 400 for determining and enhancing productivity in accordance with the present disclosure. In the embodiment shown in FIG. 4, the environment 400 includes a user 402 operating a client device 404 to remotely access an enterprise network 410 via one or more networks 406 (e.g. Internet). More specifically, in at least some implementations, communications from the client device 404 traverse an external firewall 408, an edge server 410, and an internal firewall 412 before entering the enterprise network 420. One or more biometric monitors 414 are operatively positioned proximate the user 402 to obtain biometric data regarding one or more biometric aspects of the user 402.

It will be appreciated that the environment 400 may represent a scenario wherein the user 402 may be wearing a biometric sensing device 414 (e.g. Fitbit), and may also be using a business collaboration platform on the client device 404, such as Google Apps (available from Google, Inc.) or Office 465 (available from Microsoft), and the user 402 has agreed to allow their biometric data to be collected and stored in a secure data store (e.g. one or more biometric databases 430) of the environment 400. In at least some implementations, the enterprise network 420 may be a cloud-based service.

As further shown in FIG. 4, in this embodiment, the enterprise network 420 includes a protocol head proxy server 422 that receives communications from (and may transmit communications to) the client device 404 and the one or more biometric monitors 414. One or more email and calendar servers 424 are operatively configured to exchange email information and calendar information with the protocol head proxy server 422, and to store such email information and calendar information in one or more mailbox databases 426. In at least some implementations, the email information and calendar information are provided by one or more email and calendaring applications, such as the Outlook® product commercially available from the Microsoft Corporation of Redmond, Wash.

Similarly, one or more biometric data servers 428 are operatively configured to exchange biometric information with the protocol head proxy server 422, and to store such biometric information in one or more biometric databases 430. An administrative user 425 may access and perform administrative functions on one or more of the components of the enterprise network 420 (e.g. the protocol head proxy server 422, the one or more mailbox databases 426, etc.). For example, in at least some implementations, the administrative user 425 may enter line of business data (e.g. 156 of FIG. 1), or individual goals data (e.g. 158 of FIG. 1) for performing one or more aspects of techniques and technologies disclosed herein.

With continued reference to FIG. 4, in this embodiment, the enterprise network 420 also includes a data merge server 432 that is configured to perform one or more aspects of techniques and technologies for determining and enhancing productivity as disclosed herein. For example, in at least some implementations, data merge server 432 may perform one or more aspects of the process 200 described above and depicted in FIG. 2. More specifically, in at least some implementations, the data merge server 432 may obtain individual goals data associated with the user 402, may obtain line of business data associated with a plurality of users of the enterprise network 420, may collect productivity data over a period of time regarding usage of one or more productivity tools (e.g. email and/or calendar information stored in the one or more mailbox servers 426), may collect biometric data (e.g. respiration data, heart rate, blood pressure, temperature, perspiration, skin conductivity, brain activity data, etc.), over a period of time regarding one or more of the users of the enterprise network 420 (e.g. user 402), and may analyze one or more of the productivity data, the biometric data, the line of business data, or the individual goals data. In at least some implementations, from such analyses the data merge server 432 may determine one or more productivity-related operations intended to enhance productivity.

In at least some implementations, one or more components of the enterprise network 420 (e.g. the data merge server 432) may perform one or more operational tasks 434 associated with techniques and technologies for determining and enhancing productivity. For example, in at least some implementations, one or more components of the enterprise network 420 (e.g. the data merge server 432) may conduct (or cause to be conducted) a scheduled refresh of signal data for all sources at 436. In addition, in at least some implementations, one or more components of the enterprise network 420 may enable administration (e.g. administrative user 425) or user intervention when issues occur at 438. In at least some further implementations, one or more components of the enterprise network 420 (e.g. the data merge server 432) may combine discreet sets of biometric data and productivity-related information (e.g. email and calendar information contained in the one or more mailbox databases 426) at 440.

And in at least some other implementations, one or more components of the enterprise network 420 (e.g. the data merge server 432) may compute and/or derive one or more insights from the combination of the biometric data and the productivity-related data at 442. For example, in at least some implementations, one or more insights from the combination of the biometric data and the productivity-related data may be determined using the data merge server 432, while in some implementations, such as the embodiment shown in FIG. 4, a statistical inference server 444 (e.g. Microsoft Azure®, etc.) may be tasked to statistically analyze the biometric data and the productivity-related data to determine one or more insights therefrom.

More specifically, in at least some implementations, biometric signals are uploaded from the user's one or more biometric monitors 414 to the cloud-based service (i.e. enterprise network 420). Similarly, the user's productivity signals are collected from their business collaboration platform and may be stored in the one or more mailbox databases 426. In the environment 400, the user's biometric data and collaboration data (or productivity data) (e.g. email, calendaring, etc.) are depicted as being located in the same physical datacenter (e.g. enterprise network 420), however, in alternate implementations, the biometric data and collaboration data may be stored in different locations (e.g. biometric data stored in a Microsoft storage facility such as HealthVault for Band, and collaboration data stored in a Google facility used for Google Apps). In operation, the business collaboration system (e.g. data merge server 432) accesses the biometric data from the one or more biometric databases 430 and does a data merge with the productivity data (e.g. email, calendar, etc.). In at least some implementations, line of business data (e.g. organizational structure, etc.) and/or individual goals data are also merged by the business collaboration system. When the merge occurs, new insights and goals can be created as the sum of both bio-metric and productivity data (e.g. a Total Wellness score may be computed which blends steps, sleep in hours this week, plus time spent in emails and meetings after hours). In at least some implementations, a score of “overwhelmed” can be derived from blood pressure data and time spent in meetings.

In addition, in at least some implementations, the one or more operational tasks 434 may include transformation tasks, such as the regular re-merge and refresh of insights, administration by datacenter personnel of the physical hardware and software, jobs to export and extract the combined sets of data/insights for personal and organizational analytics, a link to another solution provider such as Amazon Web Service or Microsoft Azure for statistical inference via a statistical inference engine (e.g. Hadoop by Apache Software Foundation). For example, in one possible implementation, a statistical inference such as the following question may be determined: “does number of steps taken on a daily basis predict the size of the Sales Team members total professional network size?”. Of course, in alternate implementations, a wide variety of alternate statistical inferences may be determined.

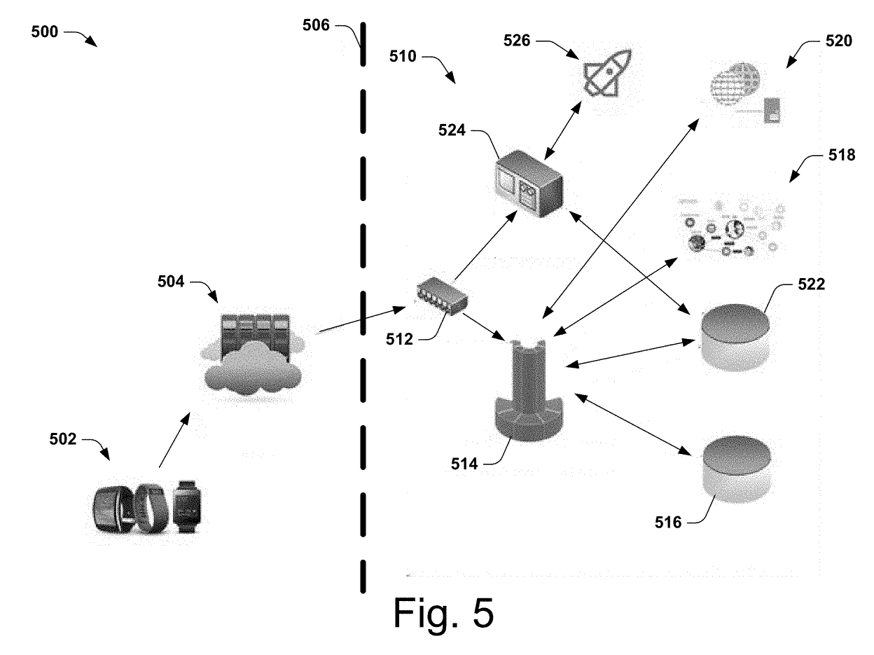

FIG. 5 shows an embodiment of a system 500 for determining and enhancing productivity. In this implementation, the system 500 includes one or more monitoring devices 502 configured to sense one or more characteristics of one or more persons. In turn, the one or more monitoring devices 502 provide information to one or more biometric services 504. In at least some implementations, the one or more biometric services 504 may include a manufacturer of at least one of the one or more monitoring devices 502, however, in other implementations, the one or more biometric services 504 may be any suitable entity that collects the information provided by the one or more monitoring devices 502 and determines from this information (e.g. by data conversion, post-processing, etc.) biometric data regarding the associated one or more persons.

As further shown in FIG. 5, the one or more biometric services 504 may provide biometric data regarding one or more persons to a message bus 512 of an enterprise network 510. In at least some implementations, the enterprise network 510 may be separated from the one or more biometric services 504 by a firewall 506. The biometric data may be pulled from the one or more biometric services 504, pushed from the one or more biometric services 504, or any suitable combination thereof.

In at least some implementations, a productivity engine 514 receives the biometric data from the message bus 512. In turn, the productivity engine 514 may store the biometric data into a long-term biometric data storage 516. More specifically, in at least some implementations, the productivity engine 514 may process the biometric data, such as by precomputing certain parameters and tagging the relevant biometric data, before storing the biometric data in the long-term biometric storage 516. A representative example of a data record that may be processed and stored in the long-term biometric storage 516 is shown below:

| | |
|---|---|
| Example: Service: Exchange, | BiometricService: |

| | |
|---|---|
| DataType: Meeting, | BiometricType: |
| time: 06/06/16 3:22 PM, | BiometricValue: |
| UserID: <guid> | BiometricDate: |
| ServiceObjectID: | BiometricUserID: |

In at least some implementations, the productivity engine 514 receives productivity data from one or more productivity applications 518 (e.g. Microsoft Office® applications suite). Similarly, the productivity engine 514 may also receive line of business data from one or more line of business services 520 (e.g. human resources department, visual studio, sales force or other CRM, administrator, etc.). In at least some implementations, the line of business data may be relatively anecdotal or case descriptive (e.g. “all of sales is not getting enough sleep,” “user x is focused while working on a bug,” “this customer seems to raise the heartbeat of all or a majority of our staff,” etc.). The productivity engine 514 may then perform one or more analyses of one or more of the productivity data, the biometric data, and the line of business data (e.g. analysis 210 of FIG. 2), and in turn may store the results of such one or more analyses into a long-term data storage 522. In at least some implementations, the results of one or more analyses determined by the productivity engine 514 may be relatively anecdotal or descriptive (e.g. “while team 1 and team 2 are communicating, they are often tense beyond normal,” “you seem to get more time focused when you set your messaging application status to ‘busy’,” “users editing documents around a project word, seem to be getting less sleep than normal, maybe the project could use some help,” etc.).

With continued reference to FIG. 5, a trends and alerting service 524 may also receive biometric data from the messaging bus 512, as well as any of the productivity data, the line of business data, or the results of one or more analyses performed by the productivity engine 514 from the long-term data storage 522. The trends and alerting service 524 may further analyze one or more aspects of the data (e.g. one or more of the productivity data, biometric data, line of business data, etc.), including by utilizing one or more logic applications that may be available from a logic application service (e.g. Microsoft Azure®). Based on such analyses, the trends and alerting service 524 may determine one or more productivity-related operations intended to enhance productivity (e.g. determining 218 of FIG. 2).

In at least some implementations, the trends and alerting service 524 analyzes one or more aspects of the data (e.g. one or more of the productivity data, biometric data, line of business data, etc.), to establish a trend of a set of biometric signals for one or more meetings associated with a user or a group of users. The result of this analysis may be referred to as a weighted average biometric score for a meeting (or meeting type), and may be used for subsequent alerts, actions or other calculations.

More specifically, in at least some implementations, the trends and alerting service 524 may receive productivity data that includes information about all meetings of a particular user, and may also receive signals of a particular biometric type from the one or more biometric services 504 associated with the meetings. The weighted average biometric score may then be computed over a desired period of time (e.g. every day, weekly, monthly, annually, etc.) which may be referred to as “timeSet”. Depending on the desired period of time, the time may be separated by hours, days, or weeks and all meetings may be assigned to an associated “ticket” (which may be referred to as “timeSetTick). The older a particular biometric signal is, the weaker the weighting assigned for that particular biometric signal (referred to as “DateWeight”). In at least some implementations, the biometric data (referred to as “Signal Value”) and the interval may differ according to the biometric data type, but the signal is averaged for the duration of the meeting (referred to as “meeting RawScore”). The number of meetings are then calculated in the desired time range (referred to as “meetingPopulation”), and then through averages the system 500 assigns each meeting within said range with a tag of meeting/biometric deviation (referred to as “MeetingDeviatedScore”). Again, the result of this analysis, referred to as a weighted average biometric score for a meeting (or meeting type), and may be used for subsequent alerts, actions or other calculations.

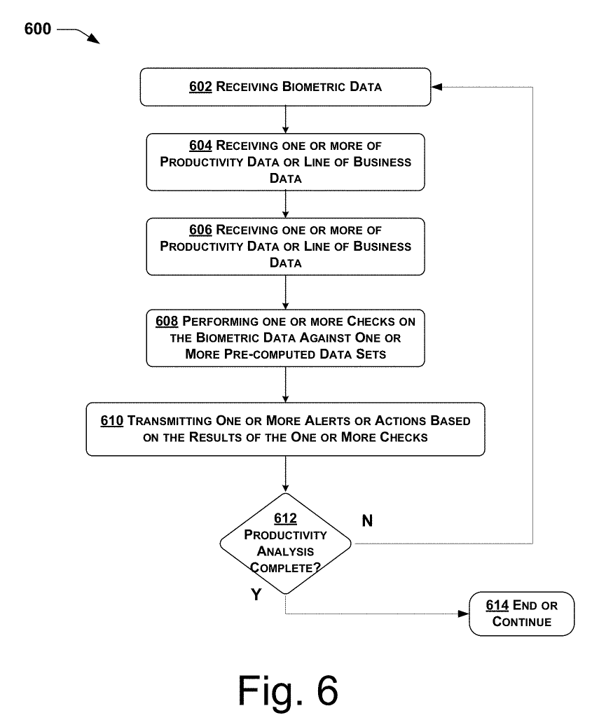

FIG. 6 shows another embodiment of a process 600 for determining and enhancing productivity. Without loss of generality, the process 600 will be described with reference to the system 500 shown in FIG. 5. In this embodiment, the process 600 includes receiving biometric data at 602. For example, the messaging bus 512 may receive biometric data from the one or more biometric services 504.

The process 600 shown in FIG. 6 further includes receiving at least one of productivity data or line of business data at 604. For example, the receiving (at 604) may include the productivity engine 514 receiving productivity data from the one or more productivity applications 518 and receiving line of business data from the line of business service 520.

The process 600 further includes performing one or more checks on the received biometric data against one or more pre-computed data sets at 608. For example, the trends and alerting service 524 may receive the biometric data from the messaging bus 512, and may receive one or more pre-computed data sets from the long-term storage 522, and may perform the one or more checks.

In the embodiment shown in FIG. 6, the process 600 further includes transmitting one or more alerts or actions based on the results of the one or more checks at 610. In various implementations, the one or more alerts or actions may be based on user input (e.g. individualized to a particular user), or may be based on one or more rules specified for a group of users (e.g. team rules, enterprise-wide rules, etc.), or may be a combination thereof. For example, in at least some implementations, the transmitting of one or more alerts or actions (at 606) may include executing a productivity software action (e.g. executing Office365 action like send email, turn off notification(s), update status, etc.). Alternately, in at least some implementations the transmitting of one or more alerts or actions (at 610) may include creating an action or trigger that may be enabled by the one or more logic applications 526 (e.g. the Microsoft Azure® logic apps).

For example, in at least some implementations, the transmitting of one or more alerts or actions (at 610) may include determining that a user's focus level has been beyond a weighted average of a predetermined threshold (e.g. “40”) for a specified period (e.g. 5 minutes), and that the user has no meetings, so the trends and alerts service 524 transmits an action to set the user's messaging application (e.g. Skype) status to a status that discourages interruption (e.g. “busy,” “focused,” “do not disturb,” etc.). Alternately, in at least some implementations, the transmitting of one or more alerts or actions (at 610) may include determining that a user's tension level is beyond a weighted average of a predetermined threshold for a specified period, and so the trends and alerts service 524 causes an action to be performed to attempt to reduce the user's stress wherein the action was previously specified by the user as a possible way to reduce the user's stress in such situations (e.g. send kitty pictures in email, etc.).

In at least some implementations, the transmitting of one or more alerts or actions (at 610) may be on both user-defined rules and enterprise-based rules. For example, in one embodiment, the trends and alerts service 524 may determine that a user is creating a meeting, and that it is the same type of meeting (e.g. based on analysis of previously-processed data from the long-term data storage 522) that has made the user stressed out in the past, or has had very poor scores in terms of communication or focus. In such a case, the trends and alerts service 524 may send an alert to the user, and ask the user whether they can make changes to this type of meeting, and/or may provide a link to “successful meeting” research to attempt to enhance the productivity of the meeting being scheduled.

With continued reference to FIG. 6, the process 600 includes determining whether the productivity analyses are complete at 612. If not, then the process 600 returns to the receiving of biometric data (at 602), and repeats the above-described operations 602-610. If productivity analyses are determined to be complete (at 612) then the process 600 ends or continues to other operations at 614. In general, techniques and technologies disclosed herein for determining and enhancing productivity may be described in the general context of computer code or machine-useable instructions, including computer-executable instructions such as program modules, being executed by a computer or other device. Generally, program modules including routines, programs, objects, components, data structures, etc., refer to code that perform particular tasks or implement particular abstract data types. Various embodiments of the invention may be practiced in a variety of system configurations, including hand-held devices, consumer electronics, general-purpose computers, more specialty computing devices, etc. In addition, various embodiments of the invention may also be practiced in distributed computing environments (e.g. cloud-based computing systems) where tasks are performed by remote-processing devices that are linked through a communications network.

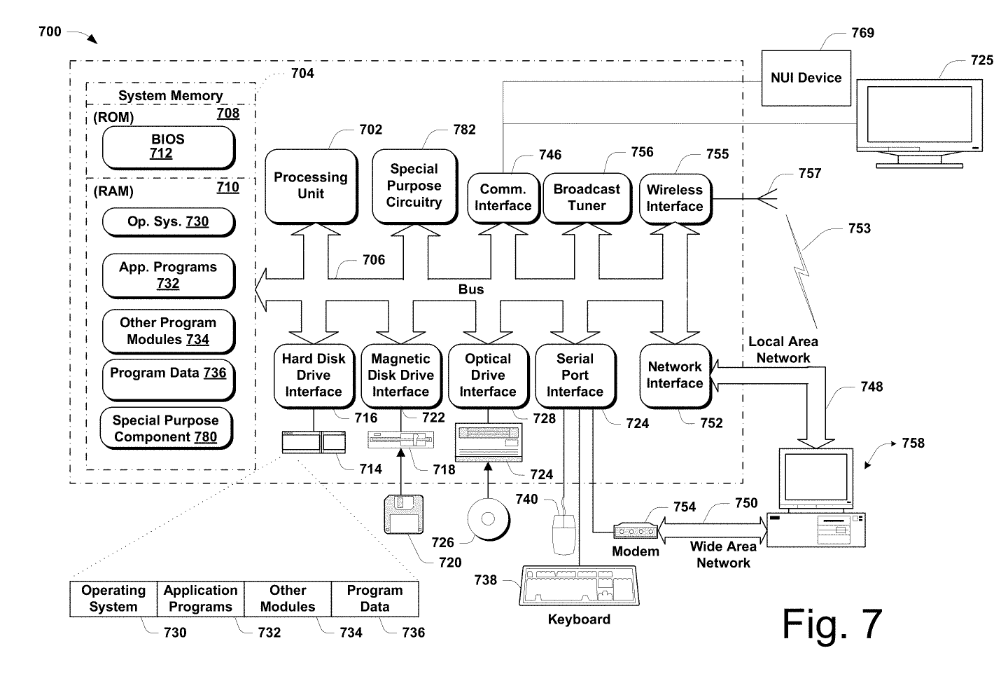

Furthermore, techniques and technologies disclosed herein for determining and enhancing productivity may be implemented on a wide variety of devices and platforms. For example, FIG. 7 shows an embodiment of a computer system 700 that may be employed for downloading visual assets for applications. As shown in FIG. 7, the example computer system environment 700 includes one or more processors (or processing units) 702, special purpose circuitry 782, memory 704, and a bus 706 that operatively couples various system components, including the memory 704, to the one or more processors 702 and special purpose circuitry 782 (e.g., Application Specific Integrated Circuitry (ASIC), Field Programmable Gate Array (FPGA), etc.).

The bus 706 may represent one or more of any of several types of bus structures, including a memory bus or memory controller, a peripheral bus, an accelerated graphics port, and a processor or local bus using any of a variety of bus architectures. In at least some implementations, the memory 704 includes read only memory (ROM) 708 and random access memory (RAM) 710. A basic input/output system (BIOS) 712, containing the basic routines that help to transfer information between elements within the system 700, such as during start-up, is stored in ROM 708.

The example system environment 700 further includes a hard disk drive 714 for reading from and writing to a hard disk (not shown), and is connected to the bus 706 via a hard disk driver interface 716 (e.g., a SCSI, ATA, or other type of interface). A magnetic disk drive 718 for reading from and writing to a removable magnetic disk 720, is connected to the system bus 706 via a magnetic disk drive interface 722. Similarly, an optical disk drive 724 for reading from or writing to a removable optical disk 726 such as a CD ROM, DVD, or other optical media, connected to the bus 706 via an optical drive interface 728. The drives and their associated computer-readable media may provide nonvolatile storage of computer readable instructions, data structures, program modules and other data for the system environment 700. Although the system environment 700 described herein employs a hard disk, a removable magnetic disk 720 and a removable optical disk 726, it should be appreciated by those skilled in the art that other types of computer readable media which can store data that is accessible by a computer, such as magnetic cassettes, flash memory cards, digital video disks, random access memories (RAMs) read only memories (ROM), and the like, may also be used.

The computer-readable media included in the system memory 700 can be any available or suitable media, including volatile and nonvolatile media, and removable and non-removable media, and may be implemented in any method or technology suitable for storage of information such as computer-readable instructions, data structures, program modules, or other data. More specifically, suitable computer-readable media may include random access memory (RAM), read only memory (ROM), electrically erasable programmable ROM (EEPROM), flash memory or other memory technology, compact disk ROM (CD-ROM), digital versatile disks (DVD) or other optical disk storage, magnetic cassettes, magnetic tape, magnetic disk storage or other magnetic storage devices, or any other medium, including paper, punch cards and the like, which can be used to store the desired information. As used herein, the term “computer-readable media” is not intended to include transitory signals.

As further shown in FIG. 7, a number of program modules may be stored on the memory 704 (e.g., the ROM 708 or the RAM 710) including an operating system 730, one or more application programs 732, other program modules 734, and program data 736 (e.g., the data store 720, image data, audio data, three dimensional object models, etc.). Alternately, these program modules may be stored on other computer-readable media, including the hard disk, the magnetic disk 720, or the optical disk 726. For purposes of illustration, programs and other executable program components, such as the operating system 730, are illustrated in FIG. 7 as discrete blocks, although it is recognized that such programs and components reside at various times in different storage components of the system environment 700, and may be executed by the processor(s) 702 or the special purpose circuitry 782 of the system environment 700.

A user may enter commands and information into the system environment 700 through input devices such as a keyboard 738 and a pointing device 740. Other input devices (not shown) may include a microphone, joystick, game pad, satellite dish, scanner, or the like. Still other input devices, such as a Natural User Interface (NUI) device 769, or user interface 725, include or involve one or more aspects of a Natural User Interface (NUI) that enables a user to interact with the system environment 700 in a “natural” manner, free from artificial constraints imposed by conventional input devices such as mice, keyboards, remote controls, and the like. For example, in at least some embodiments, the NUI device 769 may rely on speech recognition, touch and stylus recognition, one or more biometric inputs, gesture recognition both on screen and adjacent to the screen, air gestures, head and eye (or gaze) tracking, voice and speech, vision, touch, hover, gestures, machine intelligence, as well as technologies for sensing brain activity using electric field sensing electrodes (EEG and related methods) to receive inputs. In addition, in at least some embodiments, an NUI may involve or incorporate one or more aspects of touch sensitive displays, voice and speech recognition, intention and goal understanding, motion gesture detection using depth cameras (such as stereoscopic or time-of-flight camera systems, infrared camera systems, RGB camera systems and combinations of these), motion gesture detection using accelerometers/gyroscopes, facial recognition, 3D displays, head, eye, and gaze tracking, immersive augmented reality and virtual reality systems, all of which provide a more natural interface.

More specifically, in at least some embodiments, the NUI device 769 may be configured to detect one or more contacts, or one or more non-contacting gestures that are indicative of one or more characteristics, selections or actions by a user. For example, in at least some implementations, the NUI device 769 may include a non-contact gesture detection device operable to detect gestures such as a Kinect® system commercially-available from the Microsoft Corporation, a Wii® system commercially-available from Nintendo of America, Inc., a HoloLens™ system commercially-available from the Microsoft Corporation, or any of a variety of eye or gaze tracking devices, including, for example, the devices, systems, and technologies of Tobii Technology, Inc. (e.g. Pro Glasses 2, StarVR, Tobii EyeChip, Model 1750 Eye Tracker, etc.), or those of Xlabs Pty Ltd., or any other suitable devices, systems, and technologies. In this way, the NUI device 769 may be configured to detect at least one of contacts or non-contacting gestures by a user that are indicative of characteristics, selections or actions for performing operations as described above.

These and other input devices are connected to the processing unit 702 and special purpose circuitry 782 through an interface 742 or a communication interface 746 (e.g. video adapter) that is coupled to the system bus 706. A user interface 725 (e.g., display, monitor, or any other user interface device) may be connected to the bus 706 via an interface, such as a video adapter 746. In addition, the system environment 700 may also include other peripheral output devices (not shown) such as speakers and printers.

The system environment 700 may operate in a networked environment using logical connections to one or more remote computers (or servers) 758. Such remote computers (or servers) 758 may be a personal computer, a server, a router, a network PC, a peer device or other common network node. The logical connections depicted in FIG. 7 include one or more of a local area network (LAN) 748 and a wide area network (WAN) 750. Such networking environments are commonplace in offices, enterprise-wide computer networks, intranets, and the Internet. In this embodiment, the system environment 700 also includes one or more broadcast tuners 756. The broadcast tuner 756 may receive broadcast signals directly (e.g., analog or digital cable transmissions fed directly into the tuner 756) or via a reception device (e.g., via an antenna 757, a satellite dish, etc.).

When used in a LAN networking environment, the system environment 700 may be connected to the local area network 748 through a network interface (or adapter) 752. When used in a WAN networking environment, the system environment 700 typically includes a modem 754 or other means (e.g., router) for establishing communications over the wide area network 750, such as the Internet. The modem 754, which may be internal or external, may be connected to the bus 706 via the serial port interface 742. Similarly, the system environment 700 may exchange (send or receive) wireless signals 753 with one or more remote devices using a wireless interface 755 coupled to a wireless communicator 757 (e.g., an antenna, a satellite dish, a transmitter, a receiver, a transceiver, a photoreceptor, a photodiode, an emitter, a receptor, etc.).

In a networked environment, program modules depicted relative to the system environment 700, or portions thereof, may be stored in the memory 704, or in a remote memory storage device. More specifically, as further shown in FIG. 7, a special purpose component 780 may be stored in the memory 704 of the system environment 700. The special purpose component 780 may be implemented using software, hardware, firmware, or any suitable combination thereof. In cooperation with the other components of the system environment 700, such as the processing unit 702 or the special purpose circuitry 782, the special purpose component 780 may be operable to perform one or more implementations of techniques described above (e.g., example process 200 of FIG. 2, process 600 of FIG. 6, etc.).

Generally, application programs and program modules executed on the system environment 700 may include routines, programs, objects, components, data structures, etc., for performing particular tasks or implementing particular abstract data types. These program modules and the like may be executed as a native code or may be downloaded and executed, such as in a virtual machine or other just-in-time compilation execution environments. Typically, the functionality of the program modules may be combined or distributed as desired in various implementations.

In view of the disclosure of techniques and technologies for determining and enhancing productivity as disclosed herein, a few representative embodiments are summarized below. It should be appreciated that the following summary of representative embodiments is not intended to be exhaustive of all possible embodiments, and that additional embodiments may be readily conceived from the disclosure of techniques and technologies provided herein.

For example, in at least some embodiments, a system includes a processing component operatively coupled to a memory; a productivity analyzer at least partially disposed in the memory, the productivity analyzer including one or more instructions that when executed by the processing component perform operations including: receive productivity data associated with usage of one or more productivity tools by at least one user during a time period; receive biometric data associated with one or more biometric aspects of the at least one user during the time period; analyze one or more aspects of the productivity data and the biometric data; and determine based on the analysis at least one productivity-related operation intended to enhance at least one productivity metric of the at least one user.

In at least some implementations, the productivity analyzer configured to receive productivity data associated with usage of one or more productivity tools by at least one user during a time period comprises: a productivity analyzer configured to receive electronic messaging data associated with usage of an electronic messaging application by at least one user during a time period. Similarly, in at least some implementations, the productivity analyzer configured to receive productivity data associated with usage of one or more productivity tools by at least one user during a time period comprises: a productivity analyzer configured to receive electronic messaging data associated with usage of an electronic messaging application, and electronic calendaring data associated with usage of an electronic calendaring application, by at least one user during a time period.

In addition, in at least some implementations, the productivity analyzer configured to receive biometric data associated with one or more biometric aspects of the at least one user during the time period comprises: a productivity analyzer configured to receive biometric data including at least one of respiration rate, respiration volume, respiration duration, respiration pattern, heart rate, blood pressure, temperature, perspiration, skin conductivity, brain activity data, brain waves, brain temperature data, or electroencephalogram (EEG) data associated with the at least one user during the time period.

In at least some implementations, the productivity analyzer configured to analyze one or more aspects of the productivity data and the biometric data comprises: a productivity analyzer configured to determine one or more correlations between one or more aspects of the productivity data and one or more aspects of the biometric data. In other implementations, the productivity analyzer is further configured to receive line of business data, and wherein the productivity analyzer configured to analyze one or more aspects of the productivity data and the biometric data comprises: a productivity analyzer configured to determine one or more correlations between one or more aspects of the productivity data, one or more aspects of the biometric data, and one or more aspects of the line of business data.

In at least some further implementations, the productivity analyzer is further configured to receive individual goals data, and wherein the productivity analyzer configured to analyze one or more aspects of the productivity data and the biometric data comprises: a productivity analyzer configured to determine one or more correlations between one or more aspects of the productivity data, one or more aspects of the biometric data, and one or more aspects of the individual goals data. Alternately, in at least some implementations, the productivity analyzer is further configured to receive line of business data and individual goals data, and wherein the productivity analyzer configured to analyze one or more aspects of the productivity data and the biometric data comprises: a productivity analyzer configured to determine one or more correlations between one or more aspects of the productivity data, one or more aspects of the biometric data, one or more aspects of the line of business data, and one or more aspects of the individual goals data.

In addition, in at least some implementations, the productivity analyzer configured to determine based on the analysis at least one productivity-related operation intended to enhance at least one productivity metric of the at least one user comprises: a productivity analyzer configured to adjust one or more aspects of a productivity tool used by the at least one user. In further implementations, the productivity analyzer configured to determine based on the analysis at least one productivity-related operation intended to enhance at least one productivity metric of the at least one user comprises: a productivity analyzer configured to adjust one or more aspects of a displayed item displayed by a productivity tool used by the at least one user.

And in at least some implementations, the productivity analyzer configured to determine based on the analysis at least one productivity-related operation intended to enhance at least one productivity metric of the at least one user comprises: a productivity analyzer configured to provide one or more notifications including at least one of a suggestion or a recommendation to the at least one user intended to improve productivity. Alternately, in at least some other implementations, the productivity analyzer configured to determine based on the analysis at least one productivity-related operation intended to enhance at least one productivity metric of the at least one user comprises: a productivity analyzer configured to provide one or more haptic prompts to the at least one user intended to improve productivity.

In at least some further implementations, the productivity analyzer is further configured to cause the at least one productivity-related operation to be performed. For example, in at least some implementations, the productivity analyzer configured to cause the at least one productivity-related operation to be performed comprises: a productivity analyzer configured to cause at least one of: adjustment of one or more aspects of a productivity tool used by the at least one user; adjustment of one or more aspects of a displayed item displayed by the productivity tool used by the at least one user; provide one or more notifications including at least one of a suggestion or a recommendation to the at least one user intended to improve productivity; or provide one or more haptic prompts to the at least one user intended to improve productivity.

Similarly, in at least some implementations, a method at least partially implemented using one or more processing devices for determining and enhancing productivity, comprises: receiving productivity data associated with usage of one or more productivity tools by at least one user during a time period; receiving biometric data associated with one or more biometric aspects of the at least one user during the time period; analyzing using one or more processing devices one or more aspects of the productivity data and the biometric data; and determining using one or more processing devices at least one productivity-related operation at least partially based on the analysis, the at least one productivity-related operation intended to enhance at least one productivity metric of the at least one user.

In some implementations, receiving productivity data associated with usage of one or more productivity tools by at least one user during a time period comprises: receiving productivity data associated with usage of one or more productivity tools by at least one user during a time period, the productivity data including at least one of electronic messaging data, electronic mail data, electronic calendar data, word-processing data, drawing application data, spreadsheet application data, presentation application data, computer-aided design application data, social media application data, web-browsing application data, or gaming application data.

In at least some further implementations, receiving biometric data associated with one or more biometric aspects of the at least one user during the time period comprises: receiving biometric data associated with one or more biometric aspects of the at least one user during the time period, the biometric data including at least one of respiration rate, respiration volume, respiration duration, respiration pattern, heart rate, blood pressure, temperature, perspiration, skin conductivity, brain activity data, brain waves, brain temperature data, or electroencephalogram (EEG) data.

In addition, in some implementations, analyzing using one or more processing devices one or more aspects of the productivity data and the biometric data comprises: determining, using one or more processing devices, one or more correlations between one or more aspects of the productivity data and one or more aspects of the biometric data. In further implementations, analyzing using one or more processing devices one or more aspects of the productivity data and the biometric data comprises: determining, using one or more processing devices, one or more correlations between one or more aspects of the productivity data, one or more aspects of the biometric data, and one or more aspects of at least one of the line of business data or the individual goals data.

And in at least some other implementations, a system for determining and enhancing productivity, comprises: circuitry configured for receiving productivity data associated with usage of one or more productivity tools by at least one user during a time period; circuitry configured for receiving biometric data associated with one or more biometric aspects of the at least one user during the time period; circuitry configured for analyzing one or more aspects of the productivity data and the biometric data; and circuitry configured for determining at least one productivity-related operation at least partially based on the analysis, the at least one productivity-related operation intended to enhance at least one productivity metric of the at least one user.

### CONCLUSION

Those skilled in the art will recognize that some aspects of the embodiments disclosed herein can be implemented in standard integrated circuits, and also as one or more computer programs running on one or more computers, and also as one or more software programs running on one or more processors, and also as firmware, as well as virtually any combination thereof. It will be further understood that designing the circuitry and/or writing the code for the software and/or firmware could be accomplished by a person skilled in the art in light of the teachings and explanations of this disclosure.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. It will be appreciated that the embodiments of techniques and technologies described above are not exhaustive of all possible embodiments considered to be within the scope of the present disclosure, and that additional embodiments may be conceived based on the subject matter disclosed herein. For example, in alternate embodiments one or more elements or components of the techniques and technologies described above may be re-arranged, re-ordered, modified, or even omitted to provide additional embodiments that are still considered to be within the scope of the present disclosure.

Alternately, or in addition, the techniques and technologies described herein can be performed, at least in part, by one or more hardware logic components. For example, and without limitation, illustrative types of hardware logic components that can be used include Field-Programmable Gate Arrays (FPGAs), Application-Specific Integrated Circuits (ASICs), Application-Specific Standard Products (ASSPs), System-On-a-Chip systems (SOCs), Complex Programmable Logic Devices (CPLDs), etc. However, those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure.

Although the subject matter has been described in language specific to structural features and/or acts, it is to be understood that the subject matter defined in the appended claims is not necessarily limited to the specific features or acts described. Rather, the specific features and acts described above are disclosed as examples of implementing the claims and other equivalent features and acts are intended to be within the scope of the claims. The various embodiments and implementations described above are provided by way of illustration only and should not be construed as limiting various modifications and changes that may be made to the embodiments and implementations described above without departing from the spirit and scope of the disclosure.

What is claimed is:

1. A system, comprising:
a processing component operatively coupled to a memory;
a productivity analyzer at least partially disposed in the memory, the productivity analyzer including one or more instructions that when executed by the processing component perform operations including:
receive productivity data associated with usage of one or more productivity tools by at least one user during a time period;
receive biometric data associated with one or more biometric aspects of the at least one user during the time period;
analyze one or more aspects of the productivity data and the biometric data; and
determine based on the analysis at least one productivity-related operation intended to enhance at least one productivity metric of the at least one user.

2. The system of claim 1, wherein the productivity analyzer configured to receive productivity data associated with usage of one or more productivity tools by at least one user during a time period comprises:
a productivity analyzer configured to receive electronic messaging data associated with usage of an electronic messaging application by at least one user during a time period.

3. The system of claim 1, wherein the productivity analyzer configured to receive productivity data associated with usage of one or more productivity tools by at least one user during a time period comprises:
a productivity analyzer configured to receive electronic messaging data associated with usage of an electronic messaging application, and electronic calendaring data associated with usage of an electronic calendaring application, by at least one user during a time period.

4. The system of claim 1, wherein the productivity analyzer configured to receive biometric data associated with one or more biometric aspects of the at least one user during the time period comprises:
a productivity analyzer configured to receive biometric data including at least one of respiration rate, respiration volume, respiration duration, respiration pattern, heart rate, blood pressure, temperature, perspiration, skin conductivity, brain activity data, brain waves, brain temperature data, or electroencephalogram (EEG) data associated with the at least one user during the time period.

5. The system of claim 1, wherein the productivity analyzer configured to analyze one or more aspects of the productivity data and the biometric data comprises:
a productivity analyzer configured to determine one or more correlations between one or more aspects of the productivity data and one or more aspects of the biometric data.

6. The system of claim 1, wherein the productivity analyzer is further configured to receive line of business data, and wherein the productivity analyzer configured to analyze one or more aspects of the productivity data and the biometric data comprises:
a productivity analyzer configured to determine one or more correlations between one or more aspects of the productivity data, one or more aspects of the biometric data, and one or more aspects of the line of business data.

7. The system of claim 1, wherein the productivity analyzer is further configured to receive individual goals data, and wherein the productivity analyzer configured to analyze one or more aspects of the productivity data and the biometric data comprises:
a productivity analyzer configured to determine one or more correlations between one or more aspects of the productivity data, one or more aspects of the biometric data, and one or more aspects of the individual goals data.

8. The system of claim 1, wherein the productivity analyzer is further configured to receive line of business data and individual goals data, and wherein the productivity analyzer configured to analyze one or more aspects of the productivity data and the biometric data comprises:
a productivity analyzer configured to determine one or more correlations between one or more aspects of the productivity data, one or more aspects of the biometric data, one or more aspects of the line of business data, and one or more aspects of the individual goals data.

9. The system of claim 1, wherein the productivity analyzer configured to determine based on the analysis at least one productivity-related operation intended to enhance at least one productivity metric of the at least one user comprises:
a productivity analyzer configured to adjust one or more aspects of a productivity tool used by the at least one user.

10. The system of claim 1, wherein the productivity analyzer configured to determine based on the analysis at least one productivity-related operation intended to enhance at least one productivity metric of the at least one user comprises:
a productivity analyzer configured to adjust one or more aspects of a displayed item displayed by a productivity tool used by the at least one user.

11. The system of claim 1, wherein the productivity analyzer configured to determine based on the analysis at least one productivity-related operation intended to enhance at least one productivity metric of the at least one user comprises:
a productivity analyzer configured to provide one or more notifications including at least one of a suggestion or a recommendation to the at least one user intended to improve productivity.

12. The system of claim 1, wherein the productivity analyzer configured to determine based on the analysis at least one productivity-related operation intended to enhance at least one productivity metric of the at least one user comprises:
a productivity analyzer configured to provide one or more haptic prompts to the at least one user intended to improve productivity.

13. The system of claim 1, wherein the productivity analyzer is further configured to cause the at least one productivity-related operation to be performed.

14. The system of claim 1, wherein the productivity analyzer configured to cause the at least one productivity-related operation to be performed comprises:
a productivity analyzer configured to cause at least one of:
adjustment of one or more aspects of a productivity tool used by the at least one user;
adjustment of one or more aspects of a displayed item displayed by the productivity tool used by the at least one user;
provide one or more notifications including at least one of a suggestion or a recommendation to the at least one user intended to improve productivity; or
provide one or more haptic prompts to the at least one user intended to improve productivity.

15. A method at least partially implemented using one or more processing devices for determining and enhancing productivity, comprising:
receiving productivity data associated with usage of one or more productivity tools by at least one user during a time period;
receiving biometric data associated with one or more biometric aspects of the at least one user during the time period;
analyzing using one or more processing devices one or more aspects of the productivity data and the biometric data; and
determining using one or more processing devices at least one productivity-related operation at least partially based on the analysis, the at least one productivity-related operation intended to enhance at least one productivity metric of the at least one user.

16. The method of claim 15, wherein receiving productivity data associated with usage of one or more productivity tools by at least one user during a time period comprises:
receiving productivity data associated with usage of one or more productivity tools by at least one user during a time period, the productivity data including at least one of electronic messaging data, electronic mail data, electronic calendar data, word-processing data, drawing application data, spreadsheet application data, presentation application data, computer-aided design application data, social media application data, web-browsing application data, or gaming application data.

17. The method of claim 15, wherein receiving biometric data associated with one or more biometric aspects of the at least one user during the time period comprises:
receiving biometric data associated with one or more biometric aspects of the at least one user during the time period, the biometric data including at least one of respiration rate, respiration volume, respiration duration, respiration pattern, heart rate, blood pressure, temperature, perspiration, skin conductivity, brain activity data, brain waves, brain temperature data, or electroencephalogram (EEG) data.

18. The method of claim 15, wherein analyzing using one or more processing devices one or more aspects of the productivity data and the biometric data comprises:
determining, using one or more processing devices, one or more correlations between one or more aspects of the productivity data and one or more aspects of the biometric data.

19. The method of claim 15, further comprising receiving line of business data and individual goals data, and wherein analyzing using one or more processing devices one or more aspects of the productivity data and the biometric data comprises:
determining, using one or more processing devices, one or more correlations between one or more aspects of the productivity data, one or more aspects of the biometric data, and one or more aspects of at least one of the line of business data or the individual goals data.

20. A system for determining and enhancing productivity, comprising:
circuitry configured for receiving productivity data associated with usage of one or more productivity tools by at least one user during a time period;
circuitry configured for receiving biometric data associated with one or more biometric aspects of the at least one user during the time period;
circuitry configured for analyzing one or more aspects of the productivity data and the biometric data; and
circuitry configured for determining at least one productivity-related operation at least partially based on the analysis, the at least one productivity-related operation intended to enhance at least one productivity metric of the at least one user.
